(19) United States
(12) Patent Application Publication
(10) Pub. No.: US 2023/0106029 A1
(43) Pub. Date: Apr. 6, 2023

(54) METHOD OF ASSESSING THE HOMOGENEITY OF PLASTICS

(71) Applicant: ZBIGNIEW KOTULSKI ZAKLAD PRZETWÓRSTWA TWORZYW SZTUCZNYCH INGREMIO - PESZEL, Bolestaw (PL)

(72) Inventors: Wojciech KOTULSKI, Jerzmanowice (PL); Szymon SILUCH, Sosnowiec (PL)

(21) Appl. No.: 17/907,991

(22) PCT Filed: Mar. 5, 2021
(86) PCT No.: PCT/PL2021/000012

§ 371 Date: Aug. 30, 2022

(30) Foreign Application Priority Data
Feb. 3, 2020 (PL) P.433115

(51) Int. Cl. G01N 21/64; G01N 33/44; C08J 3/20; C08K 5/45; C08K 5/3475

(52) U.S. Cl. CPC G01N 21/6428; C08J 3/203; C08K 5/45; C08K 5/3475; G01N 33/442; C08J 2323/06; C08J 2323/12; G01N 2021/6439

(57) ABSTRACT

Fluorescent pigment is introduced into the plastic composition, preferably a thermoplastic, especially polyethylene or polypropylene, in the granules formation stage, in addition to possible standard components of the composite, mixed, granules are formed, and then the granules are formed into a finished building material component, and then, with the help of a known fluorimeter, the fluorescence intensity of individual parts of the surface of a plastic element is measured and the homogeneity of the material is inferred on the basis of the differences in the results of the fluorescence intensity measurements.

The subject of the invention is a method of assessing the homogeneity of plastics in the finished building material, especially in a ventilation duct.

Plastics are widely used in many areas of life, from everyday household items, through packaging and housings of many devices to fabrics or building materials. A special feature of plastics is their durability, and the necessary properties of plastic products are designed by the appropriate selection of the polymer itself and by the composition of functional additives whose task is to give new features that the polymer does not have or to strengthen the already existing properties of the plastic. In practice, the formation of the plastic composition takes place by mixing the functional additives with the plastic, e.g. in the solid phase, in the liquid phase of solutions or molten plastics. The structure of the plastics used and processed today differs significantly from that predicted more than 20 years ago, and the main reason for this is the development of more perfect polymerization methods and new technologies for mixing, compatibilization and strengthening of existing polymers.

Polymer mixtures, alloys and composites are an excellent alternative in many advanced applications, due to the methods and properties of technical plastics obtained directly by polymerization. The possibilities of obtaining materials with properties different from those of the initial polymers have been achieved thanks to the use of compounding technology, which is one of the varieties of the commonly known process of plastics extrusion.

The key issue of such compositions is the ability to reliably assess their homogeneity. The standard methods of evaluation are chemical analyses of the composition of the material and microscopic assessment of the material sample. It takes place at the stage of composite production and requires advanced research equipment. It is impossible for a construction works contractor to carry out an assessment of the homogeneity of plastic building materials in the in the field conditions, on a construction site, or in a warehouse.

The distribution of one of the components can be taken as a measure of the homogeneity of the mixture. The assessment of the uniformity of distribution of one component allows to conclude about the uniformity of distribution of the remaining components of the mixture, and the smaller the share of the assessed in the mixture, the greater the accuracy of the assessment of the homogeneity.

The essence of the method of assessing the homogeneity of plastics in the finished construction material, especially in a ventilation duct, is the fact that a fluorescent pigment is introduced into the plastic composition, preferably a thermoplastic, especially polyethylene or polypropylene, in addition to possible standard components of the composite, and mixed or possibly formed into granular form, and then the finished element of the construction material is formed, whereby, using a known fluorimeter, the fluorescence intensity of individual parts of the surface of the plastic element is measured and the homogeneity of the material is inferred from the differences in the fluorescence intensity measurements. As a fluorescent dye, an optical brightener is used, the fluorescence of which is excited by ultraviolet radiation. Preferably, the fluorescent dye is selected from the group:
2,5-thiophenediylbis(5-tert-butyl-1,3-benzoxazole)
4,4'-Bis(2-benzoxazolyl)stilbene
2-[4-[2-[4-(benzoxazol-2-yl)phenyl]vinyl]phenyl]-5-methylbenzoxazole
1,4-Bis(2-benzoxazolyl)naphthalene
4,4'-bis(2-methoxystyryl)-1,1'-biphenyl

The addition of a fluorescent dye ranges from 0.001% to 0.05% of the finished material. The fluorescent dye can be used in small amounts to be easily detected with high accuracy by radiation detectors and although it has no other functional role due to its small contribution, the properties of the composite do not change.

The method according to the invention allows for a quick assessment of the homogeneity of the plastic in the field with the use of easy-to-use, portable devices. The assessment of homogeneity allows to eliminate products with parameters insufficient for the planned applications.

### THE SUBJECT MATTER OF THE INVENTION IS SHOWN IN THE EXAMPLES

### Example I

In compounding technology, 5 g of solid 2,5-tiofenediylbis(5-tert-butyl-1,3-benzoxksazol) optical brightener are added to 5 kg of polyethylene and the extrusion granulated substance is formed. The granulated substance with the fluorescent dye is mixed with 20 kg of pure polyethylene granules and a ventilation channel is formed by extrusion. The surface of the finished ventilation duct is scanned in many places and the intensity of fluorescence is measured. The measurement results are compared with each other and on this basis it is concluded that the mass of the material is homogeneous.

### Example II

While mixing, to 100 kg of polypropylene, 1 g of solid 4,4'-bis (2-benzoxazolyl) stilbene optical brightener is added and passed to the pipe extrusion process. The plastic mixed with the dye is plasticized, compressed and extruded through the head of the pipe extruder, after which the pipe is calibrated and cooled in a calibrating cooling bath and dried. The polypropylene granules are plasticized compressed and extruded through the head of the extruders of the protective layer and a two-layer casing pipe is formed on the pipe with the dye, and then, in the crimping device — corugator, the corrugated surface of the pipe is formed under vacuum pressure, which is then calibrated under vacuum, stabilized by cooling with water in a spray bath and then dried. The inner surface of the finished pipe is scanned at multiple places and the fluorescence intensity is measured. The measurement results are compared with each other and on this basis it is concluded that the mass of the material is homogeneous.

### Example III

While mixing, to 200 kg of polypropylene, 100 g of solid optical brightener 2-[4-[2-[4(benzoxazol-2-yl) phenyl] vinyl] phenyl] -5-methylbenzoxazole is added to the process of extrusion of the air duct. The plastic mixed with the dye is plasticized, compressed and extruded through the extruder head, after which the channel is calibrated and cooled in a calibrating cooling bath and dried. The polypropylene granulate is plasticized and extruded through the extruder head of the protective layer and a casing pipe is formed on the pipe with the dye, after which the channel is vacuum calibrated, stabilized by cooling with water in a spray bath and dried. The inner surface of the finished pipe is scanned at multiple places and the fluorescence intensity is measured. The measurement results are compared with each other and on this basis it is concluded that the mass of the material is homogeneous.

1. A method of assessing the homogeneity of plastics in a finished construction material, especially in a ventilation duct, characterized in that, in addition to possible standard components of the composite, a fluorescent pigment is introduced into the plastic composition, mixed, possibly granulated, and then formed into a finished element of construction material, then, using a known fluorimeter, the fluorescence intensity of individual parts of the surface of the plastic element is measured and the homogeneity of the material is inferred on the basis of the differences in the results of the fluorescence intensity measurements.

2. The method according to claim 1, characterized in that an optical brightener is used as the fluorescent dye, the fluorescence of which is ignited by ultraviolet radiation.

3. The method according to claim 2, characterized in that 2,5-thiophenediylbis (5-tert-butyl-1,3-benzoxazoles) is used as the fluorescent dye.

4. The method according to claim 2, characterized in that 4,4'-Bis (2-benzoxazolyl) stilbene is used as the fluorescent dye.

5. The method according to claim 2, characterized in that 2-[4-[2-[4-(benzoxazol-2-yl)phenyl]vinyl]phenyl]-5-methylbenzoxazole is used as the fluorescent dye.

6. The method according to claim 2, characterized in that 1,4-Bis(2-benzoxazolyl)naphthalene is used as the fluorescent dye.

7. The method according to claim 2, characterized in that 4,4'-bis(2-methoxystyryl)-1,1'-biphenyl is used as the fluorescent dye.

8. The method according to claim 1, characterized in that 0.001% to 0.05% of a fluorescent pigment is added to the granulate.

9. The method according to claim 1, characterized in that thermoplastics are used as the plastic.

10. The method according to claim 9, characterized in that thermoplastic is polyethylene.

11. The method according to claim 9, characterized in that thermoplastic is polypropylene.
